(19) United States
(12) Patent Application Publication
(10) Pub. No.: US 2003/0053219 A1
(43) Pub. Date: Mar. 20, 2003

(54) Lens system and method

(76) Inventor: David J. Manzi, Pocono Pines, PA (US)

Correspondence Address: PENNIE AND EDMONDS, 1155 AVENUE OF THE AMERICAS, NEW YORK, NY, 100362711

(21) Appl. No.: 09/918,059

(22) Filed: Jul. 30, 2001

(51) Int. Cl. G02B 15/14; G02B 27/64; A61B 18/18

(52) U.S. Cl. 359/676; 359/557; 606/5

(57) ABSTRACT

A lens system including a first lens group having positive refractive power, a second lens group positioned forward the first lens group and having negative refractive power and including a zoom lens, and a third lens group having positive refractive power and positioned forward the first and second lens groups is disclosed. The zoom lens is movable relative to the other lenses of the lens system. The configuration and relative positioning of the lenses, along with the relative movement of the zoom lens, allows a laser transmitted through the lens system to be focused with a minimal spot size over a significant scanning field size and over a range of depths.

### FIELD OF THE INVENTION

The present invention relates to lens systems and methods, and more particularly to a zoom lens system for use in surgery, which system can efficiently and accurately focus over a range of working distances and over a wide field and range of depths.

### BACKGROUND OF THE INVENTION

Lens systems have numerous and varied applications. Lens system are especially useful and desired in certain applications, such as surgery, which involve precise positioning and operation.

For example, part of the popular LASIK (laser in-situ keratomileusis) method of laser eye surgery involves an operation that employs a metal blade to cut a portion of a patient's eye. Specifically, a spinning or vibrating microkeratome is employed to cut into the cornea of the eye to produce a “flap,” or hinged piece of corneal tissue. After the flap has been cut, it is lifted and pulled away to expose the interior of the cornea, which is then sculpted by an excimer laser to correct the vision in that eye. However, since the cornea is generally only about 0.25 to 0.5 mm thick, and the corneal flap should be cut to only about 0.15 mm thick, the precision of this cut is paramount. But performing this operation with a microkeratome is inherently imprecise, and can thus result in complications.

Because of this inherent imprecision, a technology has emerged for cutting the flap by use of a laser. A laser having a relatively long wavelength can replace the cutting operation of the microkeratome, since this laser may be focused both on and beneath the surface of the cornea, which is necessary to cut the corneal flap. For example, Intralase Corporation has developed a suitable laser, the Pulsion™ FS laser, to be used in the flap-cutting operation. The Pulsion™ FS laser has a wavelength of 1053 nm, and thus the laser will not be absorbed by the exterior surface of the cornea. Instead, it can pass through corneal tissue and be focused at a point below the cornea's outer surface without effecting the tissue. Additionally, the Pulsion™ FS laser is a high precision femto second laser, or laser with a short pulse duration in the femtosecond range, thus having the capability to provide a very precise cut to the cornea, avoiding the potential complications involved with use of the microkeratome. Such a laser could theoretically perform a precise corneal flap cut by being focused tightly through a lens system to a very small spot size across the relatively wide scanning field within the cornea and to varying depths, including a depth equal to the desired flap thickness. The laser beam could be scanned over the area of the cornea necessary to cut the flap.

Such an operation could in principle be accomplished with a lens system that does not have zooming capability. It will be appreciated, however, that absent zooming the operation would be practically difficult, since focusing the laser at different depths in the eye would require a non-zooming lens system to be moved while the patient's eye is maintained at an exact distance from the lens system during the operation to ensure a constant depth of the cut. Since patients are awake during the operation, it is generally quite difficult to prevent them from waivering from this exact distance. Alternatively, if depth of cut could be controlled by use of a zooming feature in the lens system, a lens for attaching to the eye to fix the position of the eye while the depth of cut could be varied by varying the focal distance of the laser.

But inclusion of zooming capability, along with the requirements that the laser be focused to a very small spot size across a relatively wide scanning field, complicates the design of the lens system in a way not contemplated by existing technology. Further complicating the design of such a lens system is the requirement that the working distance of the laser be sufficiently large to avoid contact of the lens system with the patient's nose. Additionally, the scanning field should be relatively flat, such that the variance in focal depth over the scan field of the cut is minimized, limiting the potential for the lenses to cut into surfaces adjacent the cornea.

Thus, there exists a need for a lens system that overcomes the above and other disadvantages of the prior art.

There also exists a need for a lens system that is employable with a laser delivery system to perform a precise cutting operation, such as the flap-cutting operation performed during laser eye surgery.

### SUMMARY OF THE INVENTION

The present invention provides a zoom lens system uniquely configured and employed to provide diffraction-limited performance over a relatively wide field of scan at a significant and varying working distance. In a specific embodiment, the present invention provides focusing precision with zooming capability to enable a laser to be focused at varying focal depths to a very small spot size over a scanning field having a diameter in a preferred embodiment of at least about 9 mm, and may be used in, for example, laser surgery. Also, the working distance in a preferred embodiment is greater than about 36 mm. Additionally, the scanning field in a preferred embodiment is advantageously substantially flat, providing a small variance in depth of focus over the entire field of scan. The zoom lens system includes one or more lenses that are movable relative to the other lenses in the system to enable focusing at varying focal depths. Advantageously, in a preferred embodiment for use in laser surgery, the present invention further includes an additional configuration of lenses, such as a lens doublet, that may be removably inserted into the zoom lens system to allow viewing through the lens system, as well as significantly increasing the working distance of the lens system, such that a user's hands or surgical or other instruments may be placed between the lens system and focused object. In another aspect the invention is a method for using the lens system in practical applications.

In one embodiment of the present invention, the lens system includes a first lens group having positive refractive power; a second lens group positioned forward the first lens group and having negative refractive power, the second lens group including one or more zoom lenses; a third lens group having positive refractive power and positioned forward the first and second lens groups; and wherein the one or more zoom lenses are movable along the along an axis, enabling a laser transmitted through the lens system to be focused at different depths, such that the lens system is capable of scanning a laser focused with a spot size of less than about 3 microns over a field having a diameter of at least about 9 mm at the different depths. The working distance of the lens system may be greater than about 36 mm. The scanning field of the lens system in a preferred embodiment has a field flatness of less than about 10 microns.

The lasers used with the lens system of the invention may be femtosecond lasers. The femtosecond laser may be the Pulsion™ FS laser. In a specific embodiment, the laser may be employed to cut a flap in the cornea of the eye.

The different depths to which the laser is transmitted through the lens system of the preferred embodiment may be focused using the one or more zoom lenses over a range from at least about +1 mm to at least about −1 mm from the nominal focal length of the lens system.

In a specific embodiment, the lens system may further include a fourth lens group movable between a first position along the axis and a second position out of alignment with the axis, the fourth lens group in the first position being placed between the second and third lens groups to increase the focal length of the lens system. The viewing field of the lens system may be increased to at least about 25 mm when the fourth lens group is in the first position. The viewing field of the lens system may be increased to at least about 30 mm when the fourth lens group is in the first position. The working distance may be increased to greater than about 100 mm when the fourth lens group is in the first position. The fourth lens group may have negative refractive power, and may include a lens doublet.

In another embodiment, a lens system has an axis and includes a first lens group having positive refractive power; a second lens group positioned forward the first lens group along the axis and having negative refractive power, the second lens group including one or more zoom lenses; a third lens group having positive refractive power and positioned forward the first and second lens groups along the axis; and wherein the one or more zoom lenses are movable along the axis such that a laser transmitted through the lens system may be focused over a range of depths while maintaining diffraction-limited performance and a numerical aperture of at least about 0.3 at a working distance of greater than about 36 mm. The laser transmitted through the lens system in a preferred embodiment can be scanned over a field having a diameter of at least about 9 mm over the range of depths. In a preferred embodiment, the ratio of the change in the depth of focus of the lens system to the movement of the zoom lens is close to 1 to 1.

The range of depths to which a laser beam is transmitted through the lens system in a preferred embodiment is at least about +1 mm to at least about −1 mm from the nominal focal length of the lens system. The laser of the lens system may be a femtosecond laser. The femtosecond laser may be the Pulsion™ FS laser.

The lens system may further include a fourth lens group movable between a position between a first position along the axis and a second position out of alignment with the axis, the fourth lens group in the first position being placed between the second and third lens groups to increase the focal length of the lens system. The lens system has a viewing field that may be increased to at least about 25 mm when the fourth lens group is in the first position. The viewing field may be increased to at least about 30 mm when the fourth lens group is in the first position. The focal length of the lens system may be increased to greater than about 100 mm when the fourth lens group is in the first position.

Another aspect of the invention is a method of performing laser eye surgery by focusing a laser with a lens system having at least one zoom lens to a predetermined position in the cornea of an eye of a patient; scanning the laser over a predetermined scanning path in the cornea to cut a corneal flap in the eye, wherein the scanning path includes a range of depths, and zooming the focus of the laser at different depths is performed using the at least one zoom lens of the lens system. The focused laser in a preferred embodiment has a spot size of less than about 3 microns. The scanning path in a preferred embodiment is associated with a scanning field having a diameter of at least about 9 mm.

The cutting of the corneal flap in accordance with the method may include focusing the laser to a specific depth within the cornea; delivering the laser to multiple spots positioned close together to form a spiral pattern, creating an incision at the specific depth; creating a stack of arc-pattened paths about the periphery of the spiral patterned cut by zooming the focus of the laser to different depths ranging from the specific depth of the incision to the surface of the cornea. In a preferred embodiment, the cutting at the specific depth has a field flatness of less than about 10 microns.

The method may further include increasing the focal depth and viewing field of the lens system so that the focal depth and viewing field are sufficiently large to enable a magnified image of the corneal flap of the eye to be viewed while having sufficient space between the lens system and the eye to allow a surgical instrument or hand to manipulate the corneal flap.

The method may further include inserting a lens group within the lens system to allow viewing of the eye through the lens system. The insertion of the lens group in a preferred embodiment provides sufficient space between the lens system and the patient's eye to allow manipulation of the corneal flap with an instrument.

The method may further include positioning a lens group within the lens system to allow viewing of the eye and to increase the working distance of the lens system to greater than about 100 mm. The positioning of the lens group within the lens system may increase the viewing field to greater than about 25 mm. The positioning of the lens group within the lens system may increase the viewing field to greater than about 30 mm. The method may further include focusing the laser at a working distance of at least about 36 mm so that the system of lenses will not interfere with the patient's nose. The different depths to which the laser may be focused is over a range from at least about +1 mm to at least about −1 mm from the nominal focal length of the lens system. The laser used in accordance with this method may be a femtosecond laser, such as the Pulsion™ FS laser.

In another aspect, the invention is a lens system for use in laser eye surgery, having a first lens group with positive refractive power; a second lens group positioned forward the first lens group and having negative refractive power and comprising a zoom lens; a third lens group having positive refractive power and positioned forward the first and second lens groups; a fourth lens group movable between a position between the second and third lens groups and a position away from the second and third lens groups such that when the fourth lens group is positioned between the second and third lens groups, the focal depth and viewing field of the lens system increases; wherein the lens group is capable of: operating at a working distance of greater than about 36 mm; scanning a field having a diameter of at least about 9 mm; focusing a laser with a spot size of less than about 3 microns; and having a numerical aperture of greater than about 0.3 over the entire scanning field; and wherein the zoom lens is movable along the principle optical axis of the lens system such that a femtosecond laser transmitted through the lens system may be focused at different depths ranging from at least about +1 mm to at least about −1 mm from the nominal focal length of the lens system.

### BRIEF DESCRIPTION OF THE DRAWING

The detailed description will be better understood in conjunction with the accompanying drawings, showing an embodiment of the invention as an example and wherein like reference characters represent like elements, as follows:

FIG. 1 is a cross-sectional side view of the first lens group of a lens system in accordance with the principles of the present invention;

FIG. 2 is a cross-sectional side view of the second lens group, including a zoom lens, of a lens system in accordance with the principles of the present invention;

FIG. 3 is a cross-sectional side view of the third lens group of a lens system in accordance with the principles of the present invention;

FIG. 4 is a cross-sectional side view of the fourth lens group of a lens system in accordance with the principles of the present invention;

FIG. 5 is a cross-sectional side view of the relative positioning of the lens groups from FIGS. 1-4 in a lens system in accordance with the principles of the present invention;

FIG. 6 is a cross-sectional side view of the relative positioning of the lens groups of FIG. 5 where the fourth lens group (not shown) is positioned out of alignment with the other lenses;

FIG. 7 is a graph showing the diffraction-limited performance of the lens system of the present invention;

FIGS. 8(a) and 8 (b) together are a table providing preferred prescription data of the lenses shown in the lens system of FIG. 6 in accordance with the present invention;

FIG. 9 is a table providing preferred prescription data of the fourth lens group from FIG. 4 and shown in FIG. 5, in accordance with the present invention; and

FIGS. 10(a) and 10(b) are tables providing preferred tolerances associated with the lens system in accordance with the present invention.

### DETAILED DESCRIPTION OF THE INVENTION

In accordance with the principles of the present invention, the lens system described herein is preferably employed to deliver and focus a laser. The separate lenses of the lens system may be produced by a company such as the Tropel Corporation or Melles Griot Inc., by providing them with the appropriate lens prescriptions, which are described herein. Companies such as Schott Glass Technologies or the Ohara Corporation can supply glass that has a Grade A industry specification, which is preferably used in the present invention to produce the lenses because of its optical quality. In this embodiment, the lens system may be employed with a laser delivery system, such as Intralase Corporation's Pulsion™ FS system (“Intralase system”), which delivers a femtosecond intrastromal laser, or with the femtosecond laser system and method described, for example, in U.S. Pat. No. 6,146,375 to Juhasz et al. (“the '375 patent”), which is incorporated herein by reference. A “femtosecond laser” refers to a laser having a pulse duration in the femtosecond range.

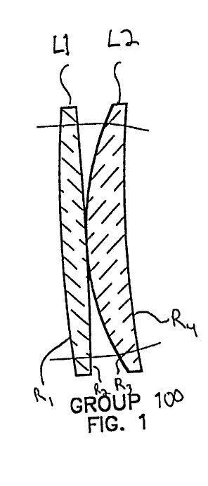

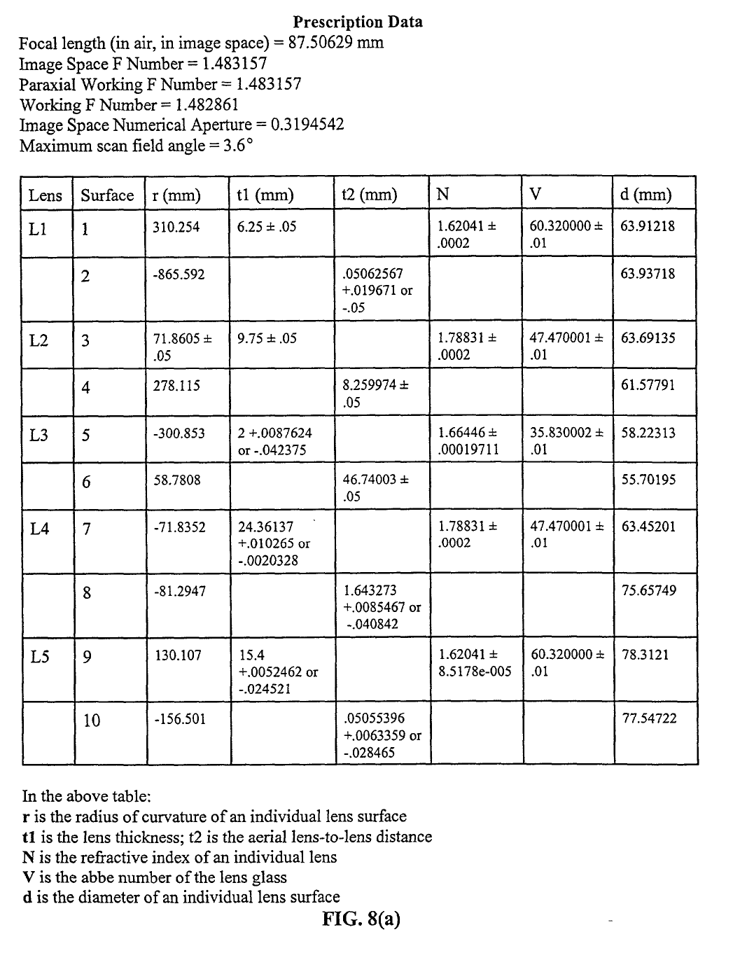

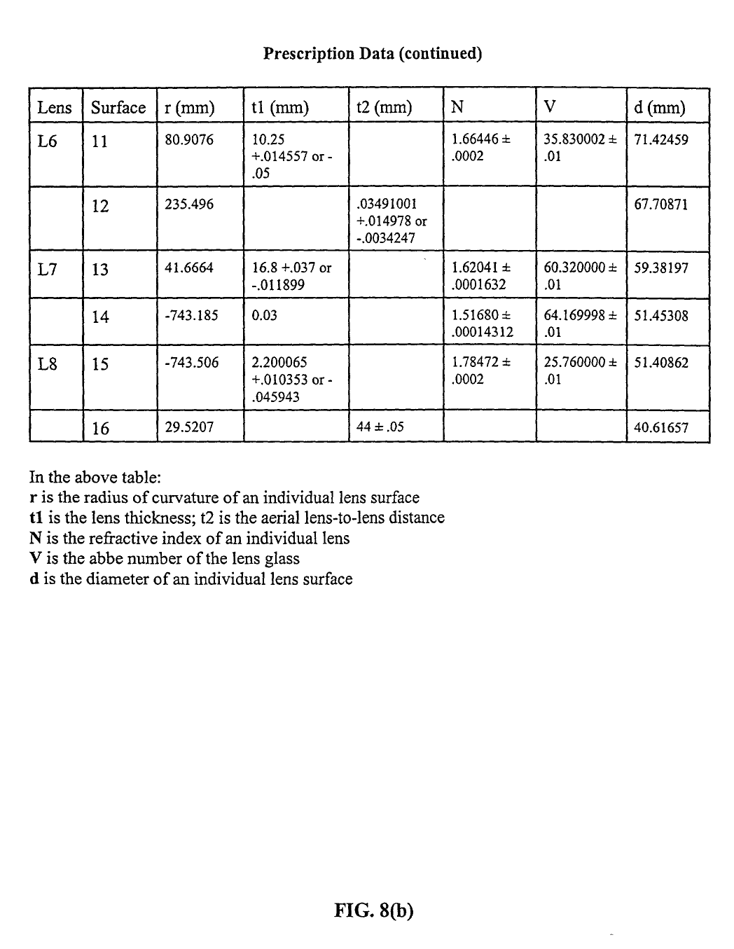

FIG. 1 shows one embodiment of a first lens group 100 of the lens system in accordance with the present invention. First lens group 100 may comprise lens L1 with radii of curvature R1 and R2, and lens L2 with radii of curvature R3 and R4. First lens group 100 preferably has positive refractive power. Specific dimensions for the radii R1 through R4 of the first lens group in a preferred embodiment are given in the table shown in FIGS. 8(a) and 8(b). The basic function, preferences, and requirements of this lens group, as well as those of the other lens groups described in this application, are those of a lens system with zooming capability. These basic functions, preferences, and requirements are known in the art, such as described in Malacara et al., Handbook of Lens Design, pp.398-405 (1994), and Kingslake, Rudolf, Lens Design Fundamentals, pp. 60-63 (1978), both of which are incorporated herein by reference.

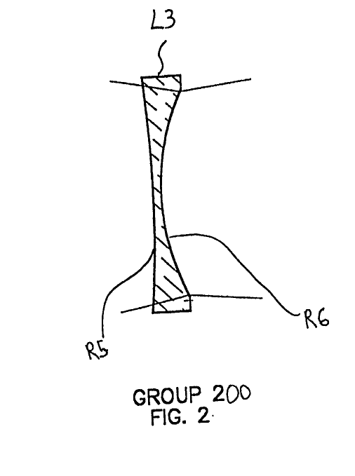

FIG. 2 shows one embodiment of a second lens group 200 of the lens system in accordance with this invention. As shown, second lens group 200 in a specific embodiment comprises single zoom lens L3. However, in alternative embodiments, more than one zoom lens may be included. The second lens group 200 preferably has negative refractive power. In accordance with this invention, zoom lens L3, which has radii of curvature R5 and R6, moves with respect to the other lenses of the system to effectuate zooming of the lens system. In a specific embodiment, the radii of curvature R5 and R6 are given in the table shown in FIGS. 8(a) and 8(b). Also, the zoom lens L3 of the second lens group 200 may be moved relative to first lens group 100, other lenses in the second group, and third lens group 300 (described below) such that light, such as a laser, transmitted through the lens system may be focused at different depths. The mechanical or other system to be employed with the lens system to accomplish accurate movement of zoom lens L3 of second lens group 200 may be provided by a company such as Carl Zeiss, Inc., or Nikon Inc.

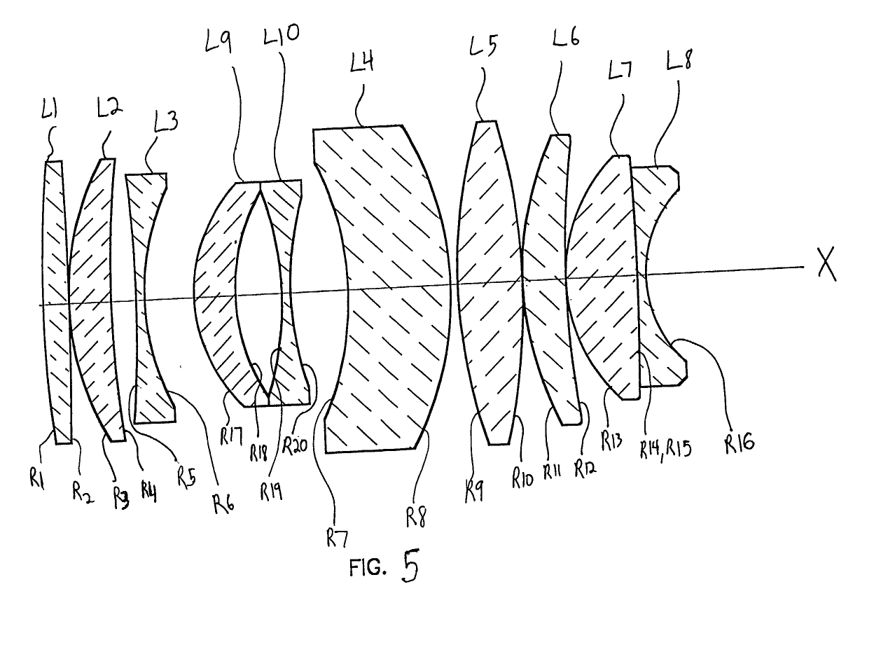

Advantageously, zoom lens L3 is configured and positioned along with the other lenses of the lens system to provide diffraction-limited performance over a wide scanning field at the varying focal lengths. Additionally, the scanning field is advantageously very flat, so that the depth of the cut over the scan field has a variance that is insignificant for operations requiring great precision, such as laser eye surgery. Preferably, zoom lens L3 is movable between first lens group 100 and third lens group 300 (and other lenses of second lens group 200, if applicable) in a direction substantially along the principal optical axis X (see FIG. 5) of the lens system. The lens system as illustrated in the embodiment of FIG. 5, and described below, advantageously provides the capability to focus at depths ranging from at least about +1 mm and −1 mm from the nominal focal length of the lens system, while still maintaining diffraction-limited performance. It will be appreciated that different focal depths can be used in alternative embodiments.

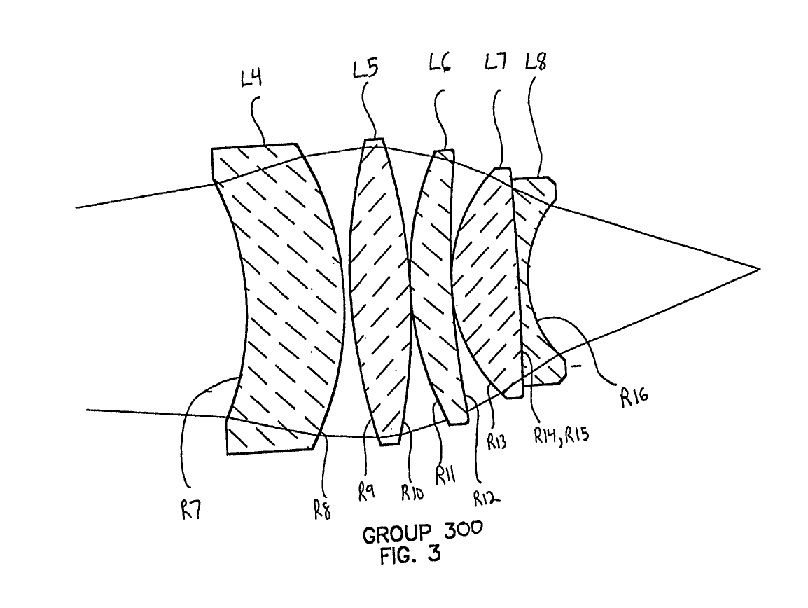

FIG. 3 shows one embodiment of a third lens group 300 of the lens system. In this embodiment, third lens group 300 may comprise five lenses L4 through L8, having radii of curvature R7 through R16, as shown in FIG. 3 and defined in a preferred embodiment in the table shown in FIGS. 8(a) and 8(b). Third lens group 300 preferably has positive refractive power. In a preferred embodiment lenses L7 and L8 are attached and have a cement interface. In the preferred embodiment defined in FIGS. 8(a) and 8(b), the interface between lenses L7 and L8 has a thickness of about 0.03 mm, as denoted in the lens thickness (t1) column in the row defining surface 14, the surface of lens L7 adjacent lens L8.

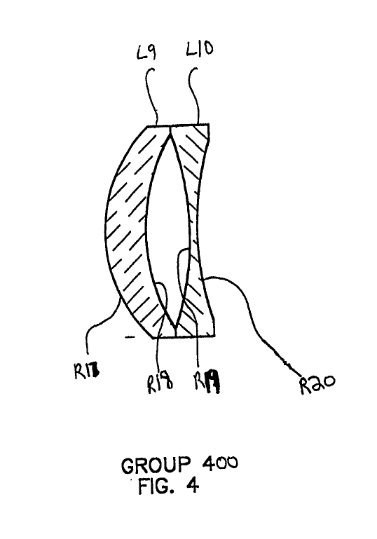

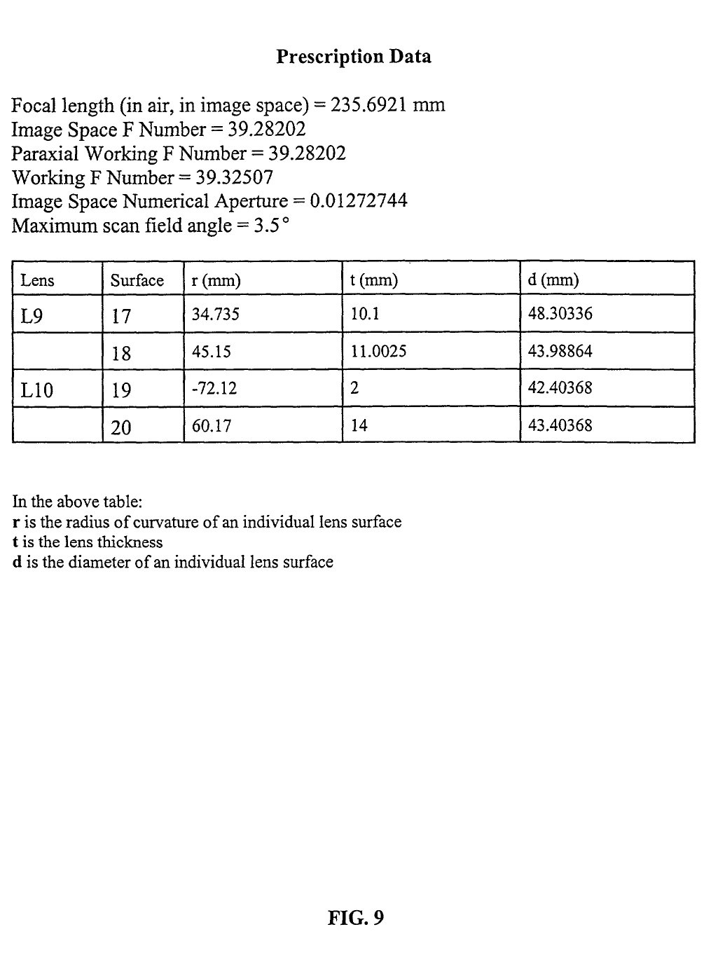

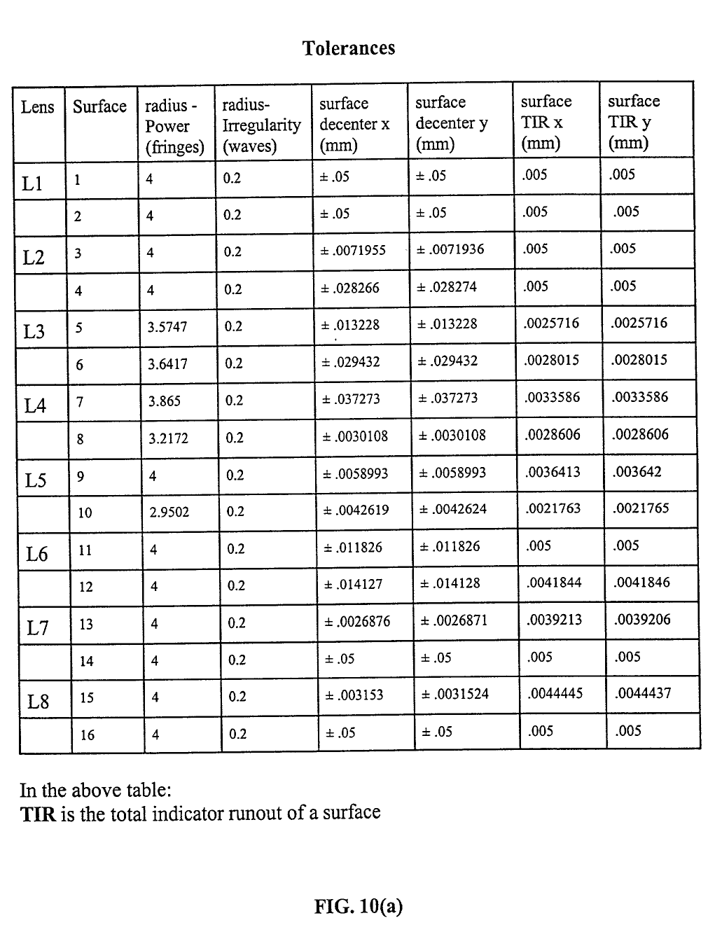

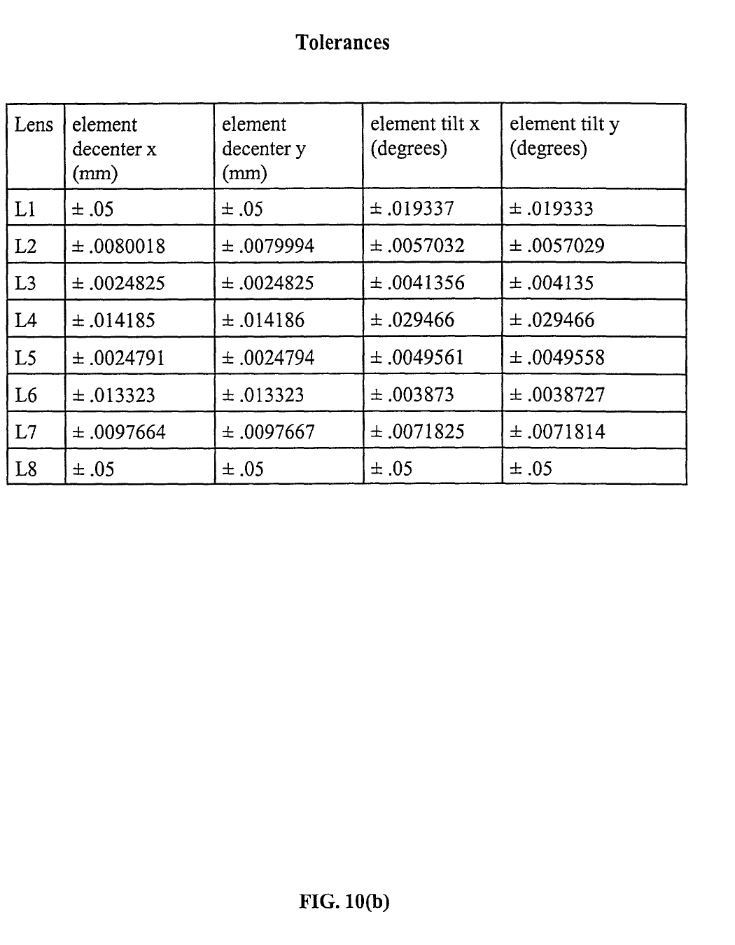

FIG. 4 shows one embodiment of a fourth lens group 400, which may be included with first through third lens groups 100 through 300, respectively, in the lens system of the present invention in a specific implementation. Fourth lens group 400 may comprise a lens doublet including lenses L9 and L10 having radii of curvature R17 through R20, as shown in FIG. 4 and defined in the preferred embodiment in the table shown in FIG. 9. The lens doublet advantageously corrects for color aberrations when a user is viewing an object through the lens system. Fourth lens group 400 preferably has negative refractive power. Fourth lens group 400, if included in the lens system, is preferably movable between two positions. One position is the “inserted” position, such that fourth lens group 400 is positioned between the second and third lens groups 200 and 300, respectively, such as shown in FIG. 5. In the inserted position, fourth lens group 400 is preferably aligned with the principal optical axis X of the lens system. In this position, the viewing field and the focal length (and thus the working distance, which is the clear distance between the object being viewed and the first optical element of the lens system) of the lens system is significantly increased. Preferably, the working distance and viewing field will be sufficiently large so that a user of the lens system will be able to view a focused, magnified image of an object while having sufficient space between the lens system and the object to allow an article, such as a surgical instrument or the user's hand, to manipulate the object without interference from the lens system. Preferably, insertion of fourth lens group 400 into the lens system increases the working distance of the lens system to greater than about 100 mm. Additionally, insertion of fourth lens group 400 will preferably widen the viewing field to a diameter of at least about 25 mm. More preferably, the viewing field will increase to at least about 30 mm in diameter. Thus, the fourth lens group 400 of the present invention provides the advantage of efficiently modifying the lens system from the operating mode (i.e., where fourth lens group 400 is not inserted), such as the laser cutting mode described below, to the viewing mode (i.e. where fourth lens group 400 is inserted) to allow quick, magnified viewing and easy manipulation of the patient or object, which is being acted upon.

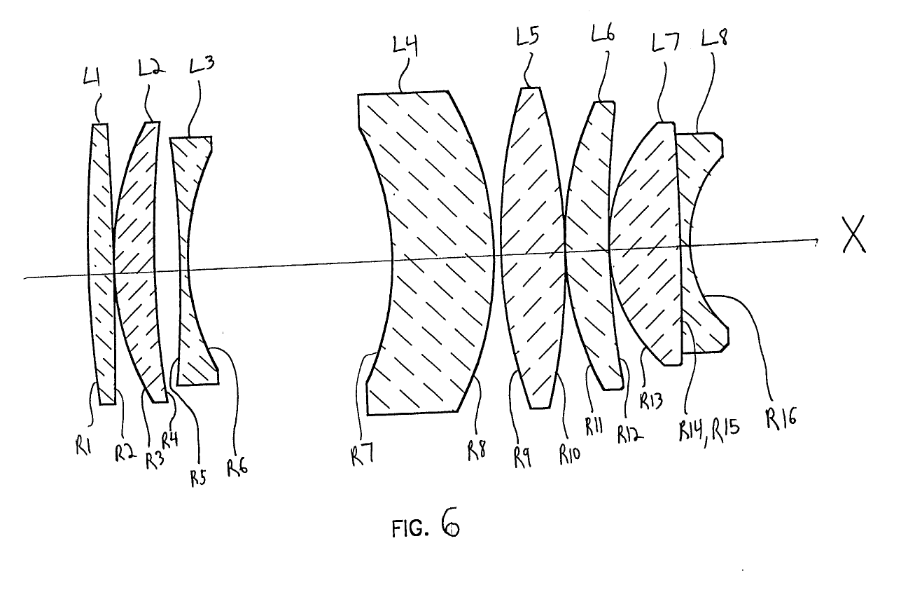

In its second position, fourth lens group 400 is positioned away from the first through third lens groups 100 through 300, respectively. In the second position, fourth lens group 400 is not in alignment with the principle optical axis X of the lens system. When fourth lens group 400 is in its second position, the operable lens system appears as illustrated in FIG. 6. In a preferred embodiment, when fourth lens group 400 is in the second position, the lens system is in the operating mode. In the operating mode, the lens system will focus an electromagnetic source of radiation, such as a femtosecond or other laser, to a small spot size over a relatively wide field of scan at a significant and varying working distance.

FIG. 5 shows an assembled lens system illustrating the relative positioning of the first, second, third, and fourth lens groups 100 through 400, respectively in a preferred embodiment. The lens system has a principle optical axis X, and the lens groups are positioned along the axis. In this embodiment, FIG. 5 shows the fourth lens group 400 when inserted into the lens system, such that the lens system is in viewing mode between the second lens group 200 and third lens group 300. In this embodiment, second lens group 200 is positioned forward first lens group 100, third lens group 300 is positioned forward second lens group 200, and fourth lens group 400 is positioned (when inserted into the lens system such that the lens system is in viewing mode) between the second lens group 200 and third lens group 300.

Where the lens system is employed with a laser delivery system, such as the Intralase system or a laser system described in the '375 patent, the fourth lens group 400, if included in the lens system, will be positioned outside the path that the laser travels when the lens system is in operating mode, as shown in FIG. 6. Thus, when the laser is delivered through the lens system, the laser will travel through the first, second, and third lens groups 100 through 300 respectively, but not fourth lens group 400. By shaping and positioning the first, second and third lens groups 100 through 300, respectively, as shown in FIG. 6, the lens system will have the capability of focusing a laser to a spot size of less than about 3 microns. The lens system will also have the capability of producing a numerical aperture of greater than about 0.3 over the entire scan field at a working distance of greater than about 36 mm. The scanning field provided by the lens configuration has a diameter of at least about 9 mm. Additionally, the ratio of the change in the depth of focus of the lens system to the movement of the zoom lens along principal optical axis X is close to 1 to 1. Thus, for example, a movement of the zoom lens of about 50 microns will correspond roughly to a 50 micron depth of field change. By keeping the depth of focus ratio close to 1 to 1, the lens system of the present invention advantageously can change its depth of focus with great precision. The reasoning is as follows: If the ratio was, for example, 5 to 1 (i.e. the depth of focus changed by five times the movement of the zoom lens), then only a 10 micron movement of the zoom lens would change the depth of field by 50 microns. Thus, the same error in movement of the zoom lens in such a configuration would result in approximately 5 times the error in depth of field focus as the present invention, making the stability and repeatability of the operation of the lens system more difficult to achieve.

In order for the lens system to actually operate with such precision, the mechanical operation of the lens system, and particularly the zoom lens of lens group 200, will need to be very accurate. The lens system will thus preferably have a stability and repeatability in the motion of the zoom lens in a direction substantially along the principal optical axis X of less than about 10 microns. Additionally, the lens system preferably positions the lenses, and moves the zoom lens, such that the lenses do not deviate from optical axis X in either de-center or tilt by more than about 5 to 10 microns. Companies such as Carl Zeiss, Inc. or Nikon, Inc. can provide mechanical systems that meet these requirements for mechanical operation. Different application-specific tolerances for the motion mechanics may be used in alternative embodiments.

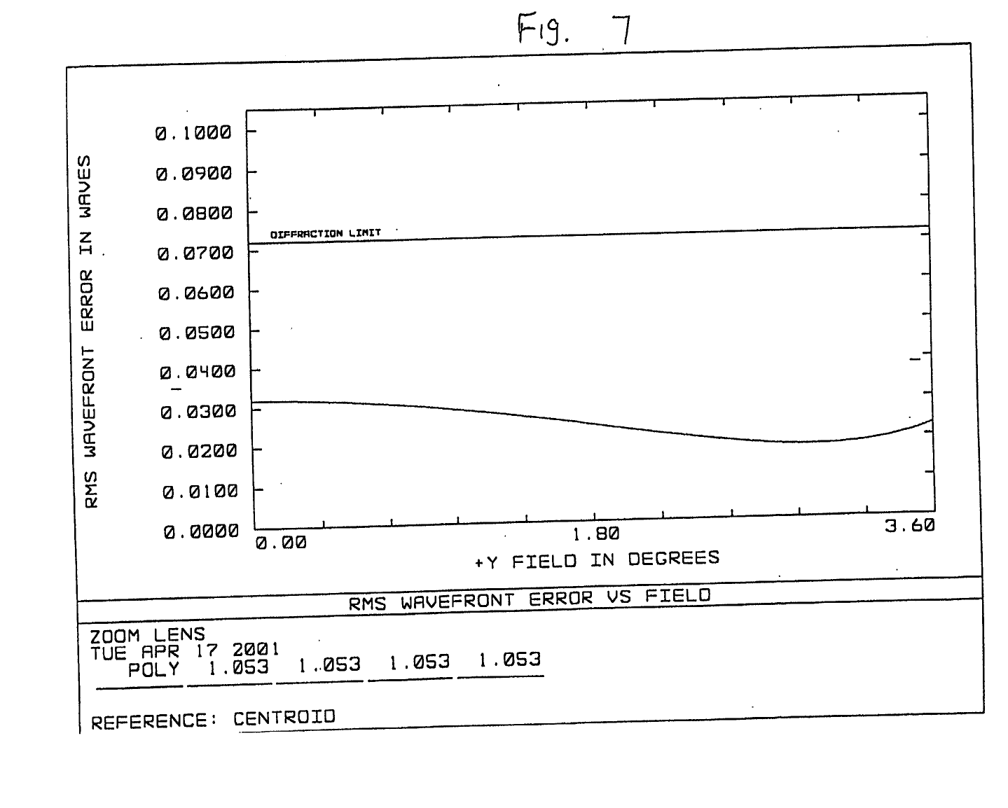

As shown in FIG. 7, the shape and configuration of the lens systems of the present invention, such as shown in FIG. 6, maintain diffraction-limited performance over the entire scanning field and range of depths. This diffraction-limited performance is illustrated in the graph in FIG. 7. FIG. 7 plots the lens system's root-mean-square (rms) wavefront error in waves versus the scanning field of the lens system of the present invention. As shown, the rms error falls below the theoretical diffraction limit over the entire ±3.60° scanning field in which the laser may be focused. Thus, for example, employment of a laser such as the Pulsion™ FS laser or a laser described in the '375 patent with the lens system of the present invention would enable cutting with a laser spot size commensurate with diffraction-limited performance of the lens system over a range of depths such as described above, and a scan field having a diameter of at least about 9 mm, and at a significant working distance (36 mm or greater) from the cutting surface. Additionally, the depth of cut over the scan field may be controlled to within less than 10 microns, ensuring that the laser will not cut unintended, adjacent surfaces in the eye.

These capabilities of the lens system advantageously provide the high level of precision required for use with a laser delivery system in eye surgery. Thus, the lens system in operating mode, as described above, may be employed with a laser system, such as the Intralase system, to focus a laser at different depths to perform a function in laser eye surgery. Preferably, a contact lens, as known in the art of laser eye surgery, is included in this embodiment for attaching to the eye to fix the position of the eye so that the depth of cut may be accurately varied by varying the focal distance of the laser. In this example, the lens system may employ the laser to perform the operation of cutting along a predetermined path in the stromal layer of the cornea of a patient's eye to form a corneal “flap,” replacing a common but less precise method of cutting the corneal flap using a microkeratome blade, such as known in the art of LASIK surgery. Preferably, this predetermined path is created by focusing the laser with the lens system to a specific depth within the cornea. The laser is then delivered to multiple “spots” positioned close together to form a spiral pattern, creating an incision underneath the cornea's surface at the specific depth. To complete the cut of the corneal flap, the focus of the laser must be zoomed by the lens system to different depths. Preferably, the laser is focused by the lens system at decreasing focal depths to create, from the depth of the spiral-patterned cut to the exterior surface of the cornea, essentially a “stack” of arc-patterned paths that are formed by focusing the laser at closely-spaced spots. The closely-spaced spots are positioned over and about the periphery of the spiral-patterned cut at each of the depths. Thus, arcs are created at different focal depths by movement of zoom lens L3 of the lens system to zoom the focus of the laser to different depths. The stacking of the arcs up to the surface form, along with the spiral-patterned cut, the corneal flap.

The precision provided by the lens system employed with a laser as described above is advantageous, since the cornea is generally about only 0.25 to 0.5 mm thick, and the corneal flap should be cut around only 0.15 mm thick. As described above, the lens system has the capability when creating the flap, as described above, to focus the laser with a small spot size at the differing depths throughout the scanning field to achieve the accuracy required in cutting the flap. Additionally, the configuration of the lenses of the present invention may enable the laser to be focused over a scan field having a flatness of less than 10 microns. Additionally, by insertion of fourth lens group 400 between second lens group 200 and third lens group 300 as described above, the focal depth and viewing field of the lens system will be sufficiently large such that a user of the lens system will be able to view a magnified image of the corneal flap of the eye, while having sufficient space between the lens system and the eye to allow a surgical instrument or the user's hand to manipulate the corneal flap.

FIGS. 8(a) and 8(b) together provide a specific example of the prescription data of a preferred embodiment for a configuration of first through third lens groups 100 through 300, respectively, used in the lens system in accordance with the present invention. In this embodiment, the lens system may be configured with the shape and relative positioning of the lenses described with respect to FIG. 6. Thus, the elements listed in FIGS. 8(a) and 8(b), e.g. focal length, are specific to the lens system where fourth lens group 400 is not aligned with the principle optical axis X.

FIG. 9 provides a specific example of the prescription data of a preferred embodiment for a configuration of the fourth lens group 400 used in the lens system in accordance with the present invention. The elements listed in FIG. 9, e.g. focal length, are specific to the lens system where, along with first through third lens groups 100 through 300, respectively, fourth lens group 400 is included and thus aligned with the principle optical axis X.

The embodiment described herein constitutes an illustrative embodiment. Other forms are possible, however. Thus, it will be clear to those skilled in the art that the present invention may be embodied in other specific forms, structures, arrangements, proportions, and with other elements, materials, and components, without departing from the spirit or essential characteristics thereof. One skilled in the art will appreciate that the invention may be used with many modifications of structure, arrangement, proportions, materials, and components and otherwise, used in the practice of the invention, which are particularly adapted to specific environments and operative requirements without departing from the principles of the present invention. The presently disclosed embodiments are therefore to be considered in all respects as illustrative and not restrictive, the scope of the invention being indicated by the appended claims, and not limited to the foregoing description.

1. A lens system having an axis, comprising:
a first lens group having positive refractive power;
a second lens group positioned forward the first lens group along the axis and having negative refractive power, the second lens group comprising one or more zoom lenses;
a third lens group having positive refractive power and positioned forward the first and second lens groups along the axis; and
wherein at least one zoom lens is movable along the axis, enabling a laser transmitted through the lens system to be focused at different depths, such that the lens system is capable of scanning a laser focused with a spot size of less than about 3 microns over a field having a diameter of at least about 9 mm at the different depths.

2. The lens system of claim 1, wherein the lens system has a nominal focal length, and wherein the different depths to which the laser is transmitted through the lens system may be focused using the one or more zoom lenses over a range from at least about +1 mm to at least about −1 mm from the nominal focal length of the lens system.

3. The lens system of claim 1, wherein the lens system has a focal length, and further comprising a fourth lens group movable between a first position along the axis and a second position out of alignment with the axis, the fourth lens group in the first position being placed between the second and third lens groups to increase the focal length of the lens system.

4. The lens system of claim 3, wherein the lens system has a viewing field that is increased to at least about 25 mm when the fourth lens group is in the first position.

5. The lens system of claim 3, wherein the lens system has a viewing field that is increased to at least about 30 mm when the fourth lens group is in the first position.

6. The lens system of claim 3, wherein the lens system has a working distance that is greater than about 100 mm when the fourth lens group is in the first position.

7. The lens system of claim 3, wherein the fourth lens group has negative refractive power.

8. The lens system of claim 6, wherein the fourth lens group comprises a lens doublet.

9. The lens system of claim 1, wherein the lens system has a working distance that is greater than about 36 mm.

10. The lens system of claim 1, wherein the lens system has a scanning field that has a field flatness of less than about 10 microns.

11. The lens system of claim 1, wherein the laser is a femtosecond laser.

12. The lens system of claim 11, wherein the femtosecond laser is used in laser eye surgery.

13. The lens system of claim 11, wherein the femtosecond laser is the Pulsion™ FS laser.

14. The lens system of claim 1, wherein the laser is employed to cut a flap in the cornea of the eye.

15. A lens system having an axis, comprising:
a first lens group having positive refractive power;
a second lens group positioned forward the first lens group along the axis and having negative refractive power, the second lens group comprising one or more zoom lenses;
a third lens group having positive refractive power and positioned forward the first and second lens groups along the axis; and
wherein at least one zoom lens is movable along the axis such that a laser transmitted through the lens system may be focused over a range of depths while maintaining diffraction-limited performance and a numerical aperture of at least about 0.3 at a working distance of greater than about 36 mm.

16. The lens system of claim 15, wherein the laser transmitted through the lens system can be scanned over a field having a diameter of at least about 9 mm over the range of depths.

17. The lens system of claim 15, wherein the lens system has a nominal focal length, and wherein the range of depths to which the laser is transmitted through the lens system is at least about +1 mm to at least about −1 mm from the nominal focal length of the lens system.

18. The lens system of claim 15, wherein the laser is a femtosecond laser.

19. The lens system of claim 18, wherein the femtosecond laser is the Pulsion™ FS laser.

20. The lens system of claim 15, wherein the lens system has a focal length, and further comprising a fourth lens group movable between a first position along the axis and a second position out of alignment with the axis, the fourth lens group in the first position being placed between the second and third lens groups to increase the focal length of the lens system.

21. The lens system of claim 20, wherein the lens system has a viewing field that is increased to at least about 25 mm when the fourth lens group is in the first position.

22. The lens system of claim 20, wherein the lens system has a viewing field that is increased to at least about 30 mm when the fourth lens group is in the first position.

23. The lens system of claim 18, wherein the lens system has a focal length, and wherein the focal length is increased to greater than about 100 mm when the fourth lens group is in the first position.

24. The lens system of claim 15, wherein the lens system has a ratio of the change in the depth of focus to the movement of the zoom lens that is close to 1 to 1.

25. A method of performing laser eye surgery, comprising:
focusing a laser with a lens system having at least one zoom lens to a predetermined position in the cornea of an eye of a patient;
scanning the laser over a predetermined scanning path in the cornea to cut a corneal flap in the eye,
wherein the scanning path includes a range of depths, and zooming the focus of the laser at different depths is performed using the at least one zoom lens of the lens system.

26. The method of claim 25, wherein the focused laser has a spot size of less than about 3 microns.

27. The method of claim 25, wherein the scanning path is associated with a scanning field having a diameter of at least about 9 mm.

28. The method of claim 25, wherein the cutting of the corneal flap comprises:
focusing the laser to a specific depth within the cornea;
delivering the laser to multiple spots positioned close together to form a spiral pattern, creating an incision at the specific depth; and
creating a stack of arc-patterned paths about the periphery of the spiral patterned cut by:
zooming the focus of the laser to different depths ranging from the specific depth of the incision to the surface of the cornea.

29. The method of claim 28, further comprising:
increasing the focal depth and viewing field of the lens system so that the focal depth and viewing field are sufficiently large to enable a magnified image of the corneal flap of the eye to be viewed while having sufficient space between the lens system and the eye to allow a surgical instrument or hand to manipulate the corneal flap.

30. The method of claim 28, wherein the cutting at the specific depth has a field flatness of less than about 10 microns.

31. The method of claim 25, further comprising:
inserting a lens group within the lens system to allow viewing of the eye through the lens system and increase the working distance of the lens system.

32. The method of claim 31, wherein the insertion of the lens group provides sufficient space between the lens system and the patient's eye to allow manipulation of the corneal flap with an instrument.

33. The method of claim 25, further comprising:
positioning a lens group within the lens system to allow viewing of the eye and to increase the working distance of the lens system to greater than about 100 mm.

34. The method of claim 33, wherein the positioning of the lens group within the lens system increases the viewing field to greater than about 25 mm.

35. The method of claim 34, wherein the positioning of the lens group within the lens system increases the viewing field to greater than about 30 mm.

36. The method of claim 35, further comprising:
focusing the laser at a working distance of at least about 36 mm so that the system of lenses will not interfere with the patient's nose.

37. The method of claim 25, wherein the lens system has a nominal focal length, and wherein the range of depths to which the laser may be focused is from at least about +1 mm to at least about −1 mm from the nominal focal length of the lens system.

38. The method of claim 25, wherein the laser is a femtosecond laser.

39. The method of claim 38, wherein the femtosecond laser is the Pulsion™ FS laser.

40. A lens system for use in laser eye surgery comprising:
a first lens group having positive refractive power;
a second lens group positioned forward the first lens group and having negative refractive power and comprising a zoom lens;
a third lens group having positive refractive power and positioned forward the first and second lens groups;
a fourth lens group movable between a position between the second and third lens groups and a position away from the second and third lens groups such that when the fourth lens group is positioned between the second and third lens groups, the focal depth and viewing field of the lens system increases;
wherein the lens system is capable of: operating at a working distance of greater than about 36 mm; scanning a field having a diameter of at least about 9 mm; focusing a laser with a spot size of less than about 3 microns; and having a numerical aperture of greater than about 0.3 over the entire scanning field; and
wherein the zoom lens is movable along the principle optical axis of the lens system such that a femtosecond laser transmitted through the lens system may be focused at different depths ranging from at least about +1 to at least about −1 mm from the nominal focal length of the lens system.

41. A lens system, substantially satisfying the following chart:

| Lens | Surface | r (mm) | t1 (mm) | t2 (mm) | N | V | d (mm) |
|---|---|---|---|---|---|---|---|
| L1 | 1 | 310.254 | 6.25 ± | | 1.62041 ± | 60.32 ± | 63.91 |
| | | | .05 | | .0002 | .01 | |
| | 2 | −865.592 | | .051 + | | | 63.94 |
| | | | | .020 or − | | | |
| | | | | .05 | | | |
| L2 | 3 | 71.8605 | 9.75 ± | | 1.78831 ± | 47.47 ± | 63.69 |
| | | | .05 | | .0002 | .01 | |
| | 4 | 278.115 | | 8.260 ± | | | 61.58 |
| | | | | .05 | | | |
| L3 | 5 | −300.853 | 2 +.01 | | 1.66446 ± | 35.83 ± 01 | 58.22 |
| | | | or − .042 | | .0002 | | |
| | 6 | 58.781 | | 46.740 ± | | | 55.70 |
| | | | | .05 | | | |
| L4 | 7 | −71.835 | 24.361 + | | 1.78831 ± | 47.47 ± | 63.45 |
| | | | .010 or − | | .0002 | .01 | |
| | | | .002 | | | | |
| | 8 | −81.295 | | 1.643 + | | | 75.66 |
| | | | | .009 or − | | | |
| | | | | .041 | | | |
| L5 | 9 | 130.107 | 15.4 + | | 1.62041 ± | 60.32 ± | 78.31 |
| | | | .005 or − | | 8.5e − 5 | .01 | |
| | | | .025 | | | | |
| | 10 | −156.501 | | .051 + | | | 77.55 |
| | | | | .006 or | | | |
| | | | | .028 | | | |
| L6 | 11 | 80.908 | 10.25 + | | 1.66446 ± | 35.83 ± | 71.42 |
| | | | .014 or − | | .0002 | .01 | |
| | | | .05 | | | | |
| | 12 | 235.496 | | .035 + | | | 67.71 |
| | | | | .015 or − | | | |
| | | | | .003 | | | |
| L7 | 13 | 41.666 | 16.8 + | | 1.62041 ± | 60.32 ± | 59.38 |
| | | | .037 or − | | .0002 | .01 | |
| | | | .012 | | | | |
| | 14 | −743.185 | 0.03 | | 1.51680 ± | 64.17 ± | 51.45 |
| | | | | | .0001 | .01 | |
| L8 | 15 | −743.506 | 2.200 + | | 1.78472 ± | 25.76 ± | 51.41 |
| | | | .010 or − | | .0002 | .01 | |
| | | | .046 | | | | |
| | 16 | 29.521 | | 44 ± .05 | | | 40.62 |
| L9 | 17 | 34.735 | 10.1 | | | | 48.30 |
| | 18 | 45.15 | 11.003 | | | | 44.0 |
| L10 | 19 | −72.12 | 2 | | | | 42.4 |
| | 20 | 60.17 | 14 | | | | 43.4 |

wherein: r is the radius of curvature of an individual lens surface;
t1 is the lens thickness;
t2 is the aerial lens−to−lens distance;
N is the refractive index of an individual lens;
V is the abbe number of the lens glass; and
d is the diameter of an individual lens surface.
wherein: r is the radius of curvature of an individual lens surface, t1 is the lens thickness; t2 is the aerial lens-to-lens distance; N is the refractive index of an individual lens; V is the abbe number of the lens glass; and d is the diameter of an individual lens surface.

42. The lens system of claim 41, wherein tolerances associated with parameters of the lens system are substantially as shown in the following tables:

| | | r: | r: | surface | surface | surface | surface |
|---|---|---|---|---|---|---|---|
| | | Power | Irregularity | decenter x | decenter y | TIR x | TIR y |
| Lens | Surface | (fringes) | (waves) | (mm) | (mm) | (mm) | (mm) |
| L1 | 1 | 4 | 0.2 | ±.05 | ±.05 | .005 | .005 |
| | 2 | 4 | 0.2 | ±.05 | ±.05 | .005 | .005 |
| L2 | 3 | 4 | 0.2 | ±.0071955 | ±.0071936 | .005 | .005 |
| | 4 | 4 | 0.2 | ±.028266 | ±.028274 | .005 | .005 |
| L3 | 5 | 3.5747 | 0.2 | ±.013228 | ±.013228 | .0025716 | .0025716 |
| | 6 | 3.6417 | 0.2 | ±.029432 | ±.029432 | .0028015 | .0028015 |
| L4 | 7 | 3.865 | 0.2 | ±.037273 | ±.037273 | .0033586 | .0033586 |
| | 8 | 3.2172 | 0.2 | ±.0030108 | ±.0030109 | .0028606 | .0028606 |
| L5 | 9 | 4 | 0.2 | ±.0058993 | ±.0059003 | .0036413 | .003642 |
| | 10 | 2.9502 | 0.2 | ±.0042619 | ±.0042624 | .0021763 | .0021765 |
| L6 | 11 | 4 | 0.2 | ±.011826 | ±.011826 | .005 | .005 |
| | 12 | 4 | 0.2 | ±.014127 | ±.014128 | .0041844 | .0041846 |
| L7 | 13 | 4 | 0.2 | ±.0026876 | ±.0026871 | .0039213 | .0039206 |
| | 14 | 4 | 0.2 | ±.05 | ±.05 | .005 | .005 |
| L8 | 15 | 4 | 0.2 | ±.003153 | ±.0031524 | .0044445 | .0044437 |
| | 16 | 4 | 0.2 | ±.05 | ±.05 | .005 | .005 |
| | element decenter x | element decenter y | element tilt x | |
|---|---|---|---|---|
| Lens | (mm) | (mm) | (degrees) | element tilt y (degrees) |
| L1 | ±.05 | ±.05 | ±.019337 | ±.019333 |
| L2 | ±.0080018 | ±.0079994 | ±.0057032 | ±.0057029 |
| L3 | ±.0024825 | ±.0024825 | ±.0041356 | ±.004135 |
| L4 | ±.014185 | ±.014186 | ±.029466 | ±.029466 |
| L5 | ±.0024791 | ±.0024794 | ±.0049561 | ±.0049558 |
| L6 | ±.013323 | ±.013323 | ±.003873 | ±.0038727 |
| L7 | ±.0097664 | ±.0097667 | ±.0071825 | ±.0071814 |
| L8 | ±.05 | ±.05 | ±.05 | ±.05 |

wherein TIR is the total indicator runout of a surface.
wherein TIR is the total indicator runout of a surface.
